(19) United States
(12) Patent Application Publication
(10) Pub. No.: US 2018/0169285 A1
(43) Pub. Date: Jun. 21, 2018

(54) HIGH-PRESSURE SPRAY OZONE DISINFECTING OF INDUSTRIAL MANUFACTURING AND STORAGE TANKS

(71) Applicant: L'Oreal, Paris (FR)

(72) Inventors: Dominick J. Bongiovi, Point Pleasant, NJ (US); Harit Patel, Clifton, NJ (US); Ashish Sinha, Skillman, NJ (US)

(73) Assignee: L'Oreal, Paris (FR)

(21) Appl. No.: 15/385,683

(22) Filed: Dec. 20, 2016

(51) Int. Cl. A61L 2/22

(52) U.S. Cl. CPC A61L 2/22; A61L 2202/13

(57) ABSTRACT

A method and system for disinfecting or sanitizing with aqueous ozone sprayed at high pressures. The method includes producing aqueous ozone having an ozone concentration of about 1 ppm to about 4 ppm. The method includes, delivering the aqueous ozone to a sprayer at a pressure of about 200 psi to about 600 psi. The method includes spraying the pressurized aqueous ozone at a surface to reduce microbe populations on the surface.

### SUMMARY

In an aspect of this disclosure, a method for disinfecting or sanitizing includes delivering aqueous ozone having an ozone concentration of about 1 ppm to about 4 ppm to a sprayer at a pressure of about 200 psi to about 600 psi; and spraying the pressurized aqueous ozone at a surface to reduce microbe populations on the surface.

In an embodiment, the method further comprises collecting the aqueous ozone after spraying and irradiating the collected aqueous ozone with UV to destroy remaining ozone.

In an embodiment, the method further comprises heating the UV irradiated water.

In an embodiment, the method further comprises rinsing a second surface with the heated and UV irradiated water.

In an embodiment, the surface is inside of a vessel.

In an embodiment, the ozone concentration is from 1 ppm to 4.5 ppm.

In an embodiment, the pressure is 200 psi to 600 psi.

In an embodiment, the pressure is 160 psi to 240 psi.

In an embodiment, the pressure is 540 psi to 660 psi.

In an embodiment, the ozone concentration is about 1 ppm to 4 ppm and the pressure is about 200 psi to about 600 psi.

In an embodiment, a temperature of the aqueous ozone is about 60° F. to about 85° F.

In an embodiment, the method comprises spraying to reduce the populations of one of Escherichia coli, Pseudomonas aeruginosa, Staphylococcus aureus, or Salmonella enterica on the surface.

In an embodiment, the method comprises reducing microbe populations by at least 2 log.

In an embodiment, the method comprises reducing microbe populations by at least 3 log.

In an embodiment, the method comprises reducing microbe populations by at least 4 log.

In an aspect of this disclosure, a system for disinfecting or sanitizing includes a storage tank containing aqueous ozone with an ozone concentration from about 1 ppm to about 4 ppm; an ozone generator connected to the storage tank; a pump connected to the storage tank, wherein the pump produces a pressure of about 200 psi to about 600 psi.

In an embodiment, the system further comprises a sprayer connected to the pump.

In an embodiment, the sprayer is a permanent fixture of a vessel.

In an embodiment, the sprayer is a removable fixture of a vessel.

In an embodiment, the storage tank has a recirculation loop.

In an embodiment, the system comprises an ozone concentration meter that measures the ozone concentration of the aqueous ozone in the storage tank.

In an embodiment, the system comprises a heater or cooler set to maintain a temperature of the aqueous ozone from about 60° F. to about 85° F.

In an embodiment, the method of disinfection and sanitization with aqueous ozone through high-pressure spray mechanisms is advantageous because it reduces the carbon footprint, and reduces utilities and performance costs; for example, the method results in the use of less water, energy, or time.

In an embodiment, aqueous ozone eliminates and replaces steam disinfection or sanitization. Steam requires 10 to 15 minutes to heat the vessel to greater than 80° C., hold for 10 minutes, and then 15 to 30 minutes to cool for safe handling and use of the vessel. In an embodiment, aqueous ozone disinfects and sanitizes the same kettle in 15 minutes or less, without heating and cooling, resulting in 50% time savings per mobile tank cleaning and disinfection or sanitization.

In an embodiment, aqueous ozone disinfection or sanitization and a final rinse step are combined into one step for additional time savings.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic illustration of a high pressure ozone disinfecting or sanitizing system;

FIG. 2 is a flow diagram of a method for high pressure ozone disinfecting or sanitizing of tanks or vessels;

FIG. 3 is a diagrammatical illustration of a vessel used in the example; and

FIG. 4 is a diagrammatical illustration of a vessel used in the example.

### DETAILED DESCRIPTION

A method and system for disinfecting or sanitizing interior or exterior surfaces of vessels, tanks, totes, or any other containers or objects with aqueous ozone at high pressure is disclosed. In an embodiment, “aqueous ozone” as used herein is water that has at least ozone dissolved therein. In an embodiment, aqueous ozone is used as an alternative to steam cleaning or sanitization. In an embodiment, ozone does not leave any residue or a carbon footprint when used for disinfecting or sanitizing. In an embodiment, the use of ozone creates energy and cost savings, reduces load on utilities, and is faster than steam sanitization. For example, when sanitizing with steam, in an embodiment steam requires 10 to 15 minutes to heat the vessel to 80° C., followed by a 10 minute hold period, and then 15 to 30 minutes are required to cool the vessel for safe handling. In an embodiment, aqueous ozone disinfects or sanitizes the same vessel in about 15 minutes or less, without the need to heat and cool resulting in about 50% time savings or more.

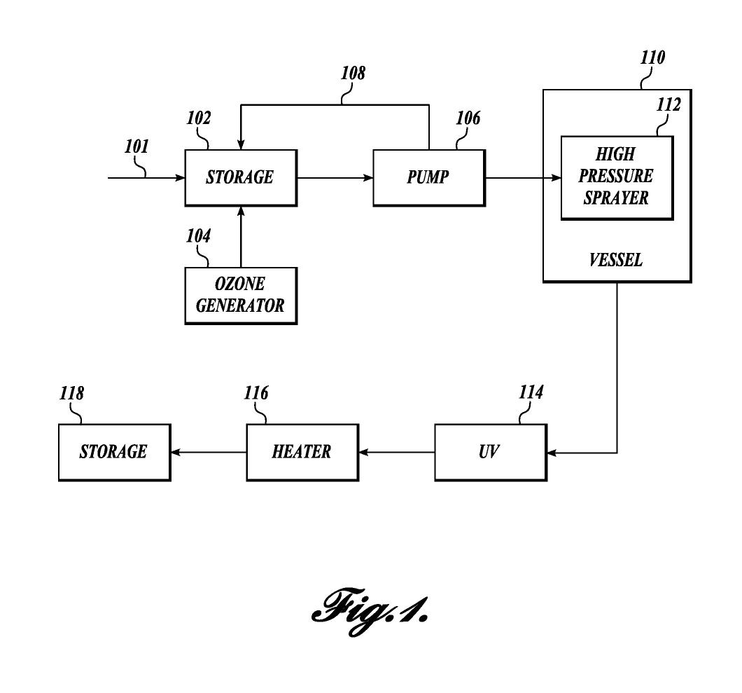

FIG. 1 is an embodiment of a high pressure aqueous ozone disinfecting or sanitizing system. In an embodiment, the system includes a storage tank 102, an ozone generator 104, a pump 106, and a high pressure sprayer 112. In an embodiment, the disinfecting or sanitizing system is piped to a vessel or tank 110 to be disinfected or sanitized.

In an embodiment, the system of FIG. 1 is used for facilities that practice “cleaning in place.” Cleaning in place or CIP is generally considered to be a process of cleaning, disinfecting, or sanitizing the interior of piping, vessels, tanks, and equipment with little to no disassembly of the equipment.

In an embodiment, the disinfecting or sanitizing system is permanently piped to a plurality of vessels. For example, in an embodiment, the disinfecting or sanitizing system is at a permanent location and is connected through a piping system to the vessel. In an embodiment where the system is stationary, the system includes valves to direct the aqueous ozone to the vessel to be disinfected or sanitized. In an embodiment, a single vessel is disinfected or sanitized at a time or a plurality of vessels are disinfected or sanitized simultaneously. Additionally, in an embodiment, a plurality of vessels are at different steps in the disinfecting or sanitizing process.

In an embodiment, the disinfecting or sanitizing system is mounted on a movable skid and transported to different areas for disinfecting or sanitizing the vessels. In an embodiment, once a vessel has been disinfected or sanitized, the disinfecting and sanitizing system is moved to a different location to disinfect or sanitize a different vessel. In an embodiment, the vessels to be disinfected or sanitized have a dedicated port which allows the insertion and removal of a high pressure sprayer. In an embodiment, the high pressure sprayer is a permanent fixture on the vessel, but, may be retracted while not in use. While the disinfecting or sanitizing system can be particularly useful for cleaning in place facilities, the invention is not thereby limited to such facilities.

In FIG. 1, in an embodiment, the storage tank 102 is any vessel that is made from a material compatible with aqueous ozone. In an embodiment, the storage tank 102 has as its purpose the storage of aqueous ozone for use in high pressure spraying of surfaces to disinfect or sanitize the surface. In an embodiment, the storage tank 102 is connected to a source of fresh water 101, such as from a municipal water supply. The water supply 101 is not particularly limiting. The incoming water 101 is combined with an ozone-containing source to raise the concentration of ozone in the water in the storage tank 102.

In an embodiment, the ozone concentration of the water in the storage tank 102 is about 1 ppm to about 4 ppm. In an embodiment, the ozone concentration of the aqueous ozone in the storage tank 102 is in the range between 1 ppm to 4 ppm. In an embodiment, the ozone concentration of the aqueous ozone in the storage tank 102 is about 2 ppm to about 4 ppm. In an embodiment, the ozone concentration of the aqueous ozone in the storage tank 102 is about 1 ppm to about 4.4 ppm. In an embodiment, the ozone concentration of the aqueous ozone in the storage tank 102 is about 3.5 ppm to about 4.5 ppm. In an embodiment, the ozone concentration of the aqueous ozone in the storage tank 102 is about 5 ppm or less. In an embodiment, the ozone concentration of the aqueous ozone in the storage tank 102 is about 6 ppm or less.

In an embodiment, the storage tank 102 has a temperature sensor used in monitoring and adjusting the temperature of the aqueous ozone in the storage tank 102. In an embodiment, the storage tank 102 has a heater to heat the aqueous ozone. In an embodiment, the storage tank 102 has a cooler to cool the aqueous ozone. In an embodiment, the storage tank 102 has exterior or interior coils to run a heating or cooling medium there-through, as the case may be, to either heat or cool the aqueous ozone. In an embodiment, the cooler is a fan driver cooler. In an embodiment, the temperature of the aqueous ozone in the storage tank 102 is heated to room temperature. In an embodiment, the temperature of the aqueous ozone in the storage tank 102 is heated in the range of from about 60° F. to about 85° F. In an embodiment, the temperature of the aqueous ozone in the storage tank is heated in the range of from about 65° F. to about 80° F. In an embodiment, the temperature of the aqueous ozone in the storage tank is heated in the range of from about 70° F. to about 75° F.

In an embodiment, the storage tank 102 includes an air vent directly to atmosphere or to an ozone destruction unit, such as a UV (ultraviolet) lamp, to reduce ozone to oxygen to prevent the escape of ozone into the atmosphere or into spaces where persons may enter.

In an embodiment, the aqueous ozone in the storage tank 102 is created by mixing ozone from the ozone generator 104 directly with the incoming fresh water before entering the storage tank 102. In an embodiment, the ozone from the ozone generator 104 is combined with the water already in the storage tank 102. In an embodiment, the ozone generator 104 introduces ozone into the water through a bubble diffuser, a venture injector, or a static mixer. In an embodiment, the storage tank 102 is operated at atmospheric pressure.

In an embodiment, the ozone generator 104 is any one of commercially available ozone generators. In an embodiment, the ozone generator 104 uses the corona discharge method for producing ozone. In an embodiment, the ozone generator 104 uses the ultraviolet light method for producing ozone. In an embodiment, the ozone generator 104 uses the cold plasma method for producing ozone. In an embodiment, the ozone generator 104 uses the electrolytic method of splitting water molecules into hydrogen, oxygen, and ozone. In an embodiment, the feed gas to the ozone generator includes either generally pure oxygen or air from the atmosphere. In an embodiment, the air is dried prior to being fed to the ozone generator 104.

In an embodiment, the production of ozone from the ozone generator 104 is controlled based on the measured ozone concentration of the aqueous ozone in the storage tank 102 with an ozone concentration meter. In an embodiment, an ozone concentration meter uses oxidation-reduction potential. In an embodiment, when the measured ozone concentration of the aqueous ozone in the storage tank 102 is below the desired concentration, a controller turns on the ozone generator 104 to raise the concentration of ozone of the aqueous ozone in the storage tank 102. Conversely, when the measured ozone concentration of the aqueous ozone in the storage tank 102 is above the desired level, the controller turns off the ozone generator 104 to not go above the desired ozone concentration. Additionally, when the measured ozone concentration of the aqueous ozone in the storage tank 102 is below the desired concentration, a controller decreases fresh water flow to the storage tank 102 to raise the concentration of ozone of the aqueous ozone in the storage tank 102, and when the measured ozone concentration of the aqueous ozone in the storage tank 102 is above the desired level, the controller increases the fresh water flow rate to the storage tank 102 to dilute the ozone concentration to not go above the desired ozone concentration. In an embodiment, whether or not the ozone generator is turned on or off as a way of controlling the ozone concentration will depend largely on the design of the ozone generator. In an embodiment, the rate of production of ozone can be increased or decreased by adjusting power or the air or oxygen feed rate, for example.

In an embodiment, the pump 106 for pumping aqueous ozone is any commercially available pump capable of generating pressure in the range of about 200 psi to about 600 psi or greater at the high pressure sprayer 112. Pressures of “psi” indicate gauge pressure (psig). In an embodiment, the pressure of about 200 psi to about 600 psi is the pressure after the pump 106. In an embodiment, the pressure is any value in the range from about 200 psi to about 600 psi or greater. In an embodiment, a pressure of about 200 psi is 180 psi to 220 psi. In an embodiment, a pressure of about 200 psi is 160 psi to 240 psi. In an embodiment, a pressure of about 600 psi is 540 psi to 660 psi. In an embodiment, a pressure of about 600 psi is 570 psi to 630 psi. The type of pump is not particularly limiting.

In an embodiment, the storage tank 102 has a recirculation loop 108. In an embodiment, the purpose of the recirculation loop 108 is to have well mixed aqueous ozone so that the ozone concentration and temperature is uniform throughout. In an embodiment, the flow of aqueous ozone in the recirculation loop 108 is provided by the high pressure pump 106. In an embodiment, the flow of aqueous ozone in the recirculation loop 108 is provided by a separate dedicated pump (not shown), which operates at a lower pressure compared to pump 106. In an embodiment, a separate dedicated pump for the recirculation loop 108 is operated continuously, where the high pressure pump 106 is operated when cleaning or sanitizing the vessels.

In an embodiment, the high pressure sprayer 112 is a sprayer wand having a spray nozzle at one end. In an embodiment, high pressure sprayer 112 includes rotating dual-nozzle sprayers, such as are sold under the name GAMMAJET™ by Alpha Laval. In an embodiment, the high pressure sprayer 112 is a hand-held sprayer with a single nozzle. In an embodiment, the high pressure sprayer 112 includes a single nozzle. In an embodiment, the high pressure sprayer 112 includes two or more than two nozzles. In an embodiment, the high pressure sprayer 112 is a permanent fixture inside of the vessel 110. In an embodiment, the high pressure sprayer 112 is a removable fixture of the vessel 110. In an embodiment, the high pressure sprayer 112 is a commercially available sprayer capable of operating at pressures of about 200 to about 600 psi, more or less. In an embodiment, the design of the high pressure sprayer is not limited, so long as the high pressure sprayer is designed for the pressures produced by the pump 106.

The vessel 100 is being used to describe a representative surface that is disinfected or sanitized. However, the system disclose herein is not limited only to disinfecting or sanitizing the inside of vessels. In other embodiments, the surface to be disinfected or sanitized includes any exterior or interior surface. In an embodiment, when disinfecting or sanitizing a vessel, the vessel 110 being disinfected or sanitized has a drain that collects the spent aqueous ozone. Spent aqueous ozone is the remains of the aqueous ozone that has been sprayed at high pressure in the vessel 110 or any other surface. In an embodiment, the piping from the drain of the vessel 110 includes a section that is transparent to ultraviolet radiation (UV). In an embodiment, a UV lamp 114 irradiates the spent aqueous ozone collected from the drain to destroy any remaining ozone. The resulting water is thus free of ozone.

In an embodiment, after the spent aqueous ozone is exposed to UV radiation by the UV lamp 114, the ozone free water is heated to a temperature of approximately 65° C. more or less with a heater 116. In an embodiment, once the ozone free water is heated to the appropriate temperature, the water can be stored in a second storage tank 118. In an embodiment, the recovered water is used for a rinse cycle in a subsequent vessel disinfection or sanitization.

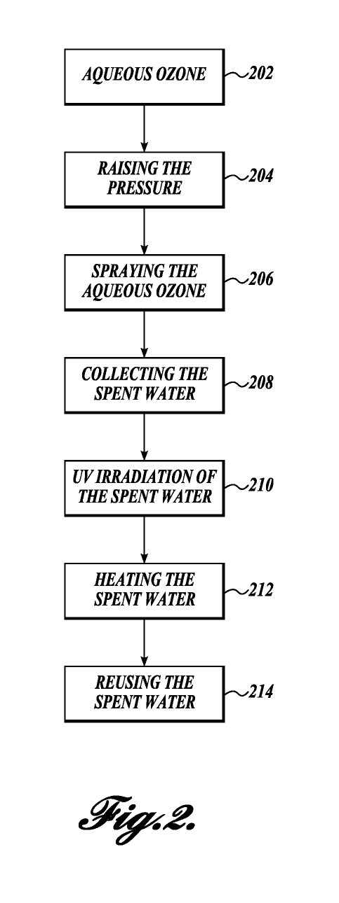

Referring to FIG. 2, in an embodiment, the system of FIG. 1 is used in a method for disinfecting or sanitizing vessels, other containers, or any surface. In an embodiment, the method includes producing aqueous ozone having an ozone concentration of about 1 ppm to about 4 ppm, block 202. In an embodiment, about 1 ppm means any value that is within the range of plus or minus 50%, such as 0.5 ppm to 1.5 ppm. In an embodiment, about 4 ppm means any value that is within the range of plus or minus 12.5%, such as 3.5 ppm to 4.5 ppm. In an embodiment, the ozone concentration is any value from about 1 ppm to about 4 pp, such as 1 ppm, 2 ppm, 3 pp, or 4 ppm. In an embodiment, the ozone concentration of the aqueous ozone is 1 ppm to 4.5 ppm or any value in between. In an embodiment, the ozone concentration of the aqueous ozone is 1 ppm to 5 ppm or any value in between.

In an embodiment, the temperature of the aqueous ozone is at room temperature. In an embodiment, the temperature of the aqueous ozone is at any one of 60° F., 61° F., 62° F., 63° F., 64° F., 65° F., 66° F., 67° F., 68° F., 69° F., 70° F., 71° F., 72° F., 73° F., 74° F., 75° F., 76° F., 77° F., 78° F., 79° F., 80° F., 81° F., 82° F., 83° F., 84° F., 85° F., or any range using any of the foregoing values as endpoints. In an embodiment, the temperature of the aqueous ozone is about 60° F. to about 85° F. In an embodiment, the temperature of the aqueous ozone is about 65° F. to 80° F. In an embodiment, the temperature of the aqueous ozone is about 70° F. to 75° F.

After block 202, the method enters block 204. In block 204, the method includes raising the pressure of the aqueous ozone in the range from about 200 psi to about 600 psi or more or less. In an embodiment, the pressure of the aqueous ozone is raised via a pump. In an embodiment, the pressure is raised to any value from 200 psi to 600 psi or more or less. In an embodiment, about 200 psi means any value that is within the range of plus or minus 10%, such as 180 psi to 220 psi. In an embodiment, about 200 psi means any value that is within the range of plus or minus 5%, such as 190 psi to 210 psi. In an embodiment, about 600 psi means any value that is within the range of plus or minus 10%, such as 540 psi to 660 psi. In an embodiment, about 600 psi means any value that is within the range of plus or minus 5%, such as 570 psi to 630 psi. In an embodiment, the pressure of the aqueous ozone is raised from 180 to 660 or any value in between.

After block 204, the method enters block 206. In block 206; the method includes spraying the pressurized aqueous ozone at a surface to reduce microbe populations on the sprayed surface. In an embodiment, the primary mode of reducing microbe populations is through ozone and the strong oxidizing effect, and a secondary mode is through mechanical force of the high pressure spray. That is, the majority of microbe population reduction is through the action of ozone not mechanical force. In an embodiment, a minority portion of microbe population reduction is through mechanical force.

In an embodiment, microbes include any unicellular or multicellular organisms. In an embodiment, the method reduces the population of viruses or bacteria or both viruses and bacteria on surfaces sprayed with the aqueous ozone at high pressure. In an embodiment, the organisms removed do not need to be pathogenic. The surface is also not particularly limiting. In an embodiment, the surface is anywhere on the inside of a vessel, tanks, or other container. In an embodiment, a surface is an exterior surface, such as countertops, conveyors, tools, cookware, surgical implements, and the like. In an embodiment, spraying the aqueous ozone at the pressures described herein reduces microbe populations by several log. In an embodiment, representative microbes that are reduced via spraying of high pressure aqueous ozone include, but are not limited to, Escherichia coli ATCC 25922, Pseudomonas aeruginosa ATCC 15442, Staphylococcus aureus ATCC 6538, and Salmonella enterica ATCC 10708. In an embodiment, the microbe population on the treated surface is reduced by 2 to 5 log. In an embodiment, the microbe population on the treated surface is reduced by at least 2 log. In an embodiment, the microbe population on the treated surface is reduced by at least 3 log. In an embodiment, the microbe population on the treated surface is reduced by at least 4 log. In an embodiment, the microbe population on the treated surface is reduced by at least 5 log.

In an embodiment, log reduction is calculated according to ASTM E2315-03 (2008). Log₁₀ reduction (LR)=mean log₁₀ (initial microbial population)−mean log₁₀ (surviving microbial population)

Where:
Initial microbial population=numbers control (untreated) population
surviving microbial population=ozone-treated population

In an embodiment, the duration of spraying is about 5 to about 15 minutes, or more or less. It is to be appreciated that the spraying time to achieve a certain log reduction is inversely related to the ozone concentration. That is, aqueous ozone with higher ozone concentration require less time to achieve the same reduction with aqueous ozone of lower ozone concentration. In an embodiment, the spraying time is at least or at most 1, 2, 3, 4, 5, 6, 7, 8, 9, 10, 11, 12, 13, 14, or 15 minutes or any range between any of the foregoing values. In an embodiment, the spraying time is more than 15 minutes. The aqueous ozone flow rate will generally be dictated by the nozzle design. Generally, for flow at about 200 psi, in an embodiment, the flow will be about 13 to about 16 gallons per minute. For flow at about 600 psi, the flow will be about 4 to 5 gallons per minute. However, the aqueous ozone flow rates are not thereby limited.

After block 206, the method enters block 208. In an embodiment, block 208 is optional. In block 208, the method includes collecting the spent aqueous ozone. Spent aqueous ozone is the aqueous ozone after it has been sprayed at or on the surface. In an embodiment, the spent aqueous ozone is collected by installing a drain in the vessel being sprayed. Spent aqueous ozone contains less ozone compared to the aqueous ozone that is sprayed. In an embodiment, the spent aqueous ozone contains less than 1 ppm ozone. In an embodiment, the spent aqueous ozone contains less than 0.5 ppm ozone. In an embodiment, where the surface to be sprayed is not the interior of a vessel, the spent ozone is collected by placing a catch basin underneath or around the surface to be sprayed. In such case, the catch basin has a drain to direct the collected spent aqueous ozone. In another embodiment, vacuum trucks are used to collect the spent aqueous ozone that spills on the floor. In another embodiment, hoods are placed over the surface to be sprayed. The hoods are connected to a vacuum hose that vents the ozone through a UV destruction unit before venting to atmosphere.

After block 208, the method enters block 210. In an embodiment, block 210 is optional. In block 210, the method includes irradiating the spent aqueous ozone with UV (ultraviolet) to destroy any remaining ozone and producing ozone free water. Similarly, any other spent aqueous ozone or vented ozone collected in catch basis, vacuum trucks, hoods or the like is treated with UV for destruction prior to re-use or release.

After block 210, the method enters block 212. In an embodiment, block 212 is optional. In block 212, the method includes heating the UV irradiated spent aqueous ozone.

After block 212, the method enters block 214. In an embodiment, block 214 is optional. In block 214, the method includes re-using the ozone free water after heating and ozone destruction. In an embodiment, the water is used for rinsing a second vessel to be disinfected or sanitized.

In an embodiment, the system is used for disinfecting or sanitizing the inside surface of a vessel, tank, pipe, or other equipment. The system and method are not just limited to disinfecting or sanitizing the inside of vessels. In an embodiment, the system is used for disinfecting or sanitizing an exterior surface of equipment or tools or any other object that is desired to be disinfected or sterilized.

In an embodiment, the system and method of disinfecting or sanitizing with aqueous ozone at high pressure has application in cosmetic manufacturing facilities. In an embodiment, the system and method of disinfecting or sanitizing with aqueous ozone at high pressure has application in food and food preparation facilities. In an embodiment, the system and method of disinfecting or sanitizing with aqueous ozone at high pressure has application in pharmaceutical manufacturing facilities. In an embodiment, the system and method of disinfecting or sanitizing with aqueous ozone at high pressure has application in a hospital. The foregoing list is not meant to be limiting of the applications that are possible with the high pressure aqueous ozone system described herein.

In an embodiment, the system and method of disinfecting or sanitizing with aqueous ozone at high pressure obtains a sustainable and environmentally friendly cleaning and sanitizing method compared to traditional steam and/or chemical disinfection or sanitization. Ozone quickly reduces into oxygen without any byproducts after use. In an embodiment, the spent water after spraying is recaptured and reused, therefore, there is zero net water consumption.

The method of disinfection or sanitization with aqueous ozone through high pressure sprayers is advantageous because it reduces the carbon footprint and utilities. Overall, the method results in less water consumption, less energy consumption, and reduced disinfecting or sanitizing time compared to steam.

### Example

1. This example describes a test to evaluate the efficacy of aqueous ozone supplied at high pressures for disinfection of vessels. Organisms that were evaluated included Escherichia coli ATCC 25922, Pseudomonas aeruginosa ATCC 15442, Staphylococcus aureus ATCC 6538, and Salmonella enterica ATCC 10708.

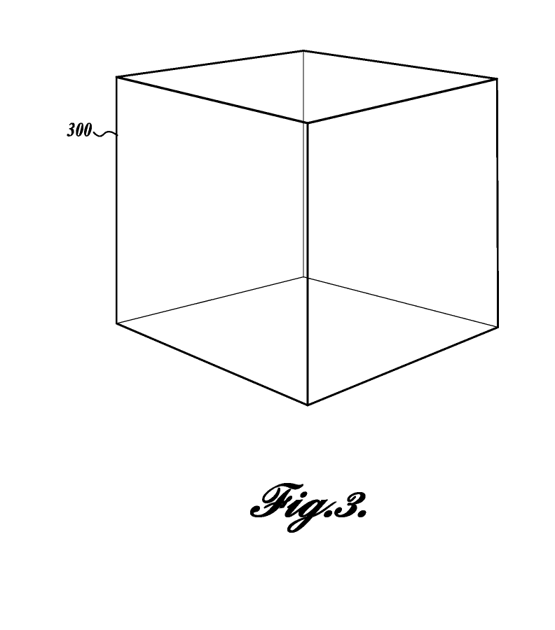

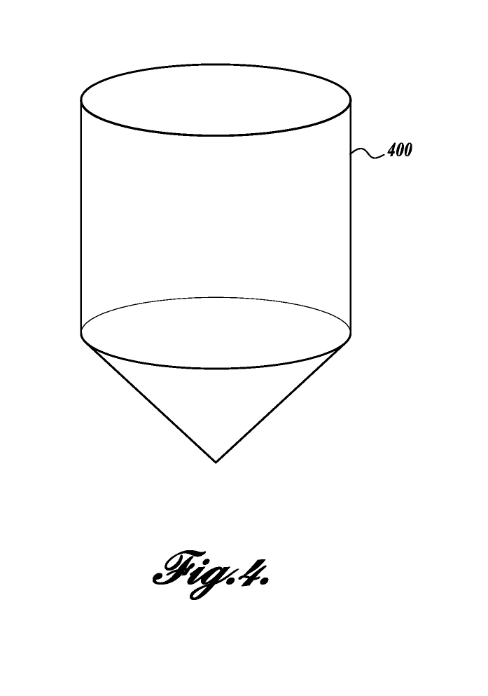

2. Two vessel configurations were used to evaluate log reductions of the subject organisms exposed to aqueous ozone sprayed at high pressure. The vessels were fitted with a Gammajet™ IX sprayer with a design flow of 14-16 gpm and a pressure of 180-200 psi. The target aqueous ozone temperature was 70° F. Carrier coupons were placed in likely dead zones where the Gammajet™ sprayer is less likely to reach. FIGS. 3 and 4 depict a cube vessel 300 representative of a square tote (hereinafter “cube”) and a cone bottom vessel 400 (hereinafter “cone”), respectively. Carrier coupon placement for the cube 300 included an upper and lower corner, an upper, middle, and lower area of the sidewall, and the bottom and top walls. Carrier coupon placement for the cone included an upper, middle, and lower area of the cylinder, the top wall, and the apex of the cone.

3. The ozone generator used in this example was a Genesis™, model Q45HCD-25G-PRO. Ozone monitors were used to measure the influent ozone concentration of the aqueous ozone, gaseous ozone at the surface, and the spent aqueous ozone after each trial.

4. The Gammajet™ sprayer head was centered in the top wall of the cube 300 and cone 400. However, different orifice sizes were used. A smaller orifice of 0.06″ was used on the cube 300 and a larger orifice size of 0.1435″ was used on the tank 400.

5. Three replicate coupons with the biofilm consortium or dried smear were placed at each of the locations described above. The dried smears and biofilm consortiums were prepared in a manner consistent with the protocols specified in AOAC and ASTM methodology.

6. Test Replicates

Each test position in the cube 300 and cone 400 held three coupon replicates in close proximity to each other. Each test run included three untreated coupon replicates as controls. Within a given test run, the samples and controls were prepared, eluted, and enumerated in a paired fashion in order to control for differences in coupon exposure to desiccating conditions. This was done to ensure that log reductions calculated for a given treatment condition or vessel location would be attributed solely to the treatment. The samples were plated in duplicate on four selective media to enumerate the four challenge organisms.

7. Calculation of Log reduction

Log reduction was calculated according to ASTM E2315-03 (2008). Log₁₀ reduction (LR)=mean log₁₀ (initial microbial population)−mean log₁₀ (surviving microbial population)

Where:
Initial microbial population=numbers control (untreated) population
surviving microbial population=ozone-treated population

8. Test Conditions

TABLE 1
Test conditions per each trial.

| | | Water | Spray | Water | Flow | Influent. | Effluent |
|---|---|---|---|---|---|---|---|
| | | Temp | Time | Pressure | rate | O₃ (ppm, | O₃ (ppm, |
| Test No. | Vessel/Preparation | (° F.) | (Minutes) | (psi) | (gpm) | Average) | Average) |
| 1 | Cone/Biofilm | 72.7 | 10 | 225 | 13.8 | 4.28 | 0.24 |
| 2 | Cone/Biofilm | 62.9 | 15 | 199 | 14.5 | 4.12 | 0.52 |
| 3 | Cube/Biofilm | 77.1 | 10 | 600 | 4.3 | 3.79 | 0.15 |
| 4 | Cube/Biofilm | 76.5 | 15 | 606 | 4.5 | 4.24 | 0.29 |
| 5 | Cone/Smear | 68.9 | 5 | 213 | 13.9 | 4.03 | 0.17 |
| 6 | Cone/Smear | 62.3 | 7 | 199 | 14.5 | 4.20 | 0.46 |
| 7 | Cube/Smear | 75.4 | 5 | 600 | 4.2 | 4.16 | 0.22 |
| 8 | Cube/Smear | 73.5 | 7 | 603 | 4.1 | 4.22 | 0.27 |
| | Average | 71.16 | | | | 4.13 | 0.29 |

20. Results

| | Log Reduction (min and max out of tested locations) | | | | | | | |
|---|---|---|---|---|---|---|---|---|
| Test | S. enterica | | E. coli | | P. aeruginosa | | S. aureus | |
| No. | minimum | maximum | minimum | maximum | minimum | maximum | minimum | maximum |
| 1 | 3.35 | >4.49 | 1.83* | >5.08 | 3.17 | >4.34 | >4.27 | >4.36 |
| 2 | 2.70 | >4.17 | 2.70 | >4.62 | 1.08* | >2.83 | >4.85 | >5.20 |
| 3 | >4.84 | >4.94 | >4.81 | >5.18 | >3.96 | >4.47 | >4.66 | >5.06 |
| 4 | 4.67 | >5.52 | 4.20 | >6.01 | 3.16 | >4.70 | >4.20 | >4.20 |
| 5 | 5.55 | 5.55 | 5.68 | 5.88 | 5.32 | 5.32 | 5.80 | 5.80 |
| 6 | 5.64 | 5.64 | 5.87 | 5.87 | 5.46 | 5.46 | 5.85 | 5.85 |
| 7 | >5.59 | >5.59 | >5.77 | >5.77 | >4.71 | >4.71 | >5.44 | >5.44 |
| 8 | >5.87 | >5.87 | >5.71 | >5.71 | >5.58 | >5.58 | >5.67 | >5.67 |

*denotes value below industry standard, all other values meet or exceed industry standard
Values marked with “>” indicates a “total kill” where there was no measureable remaining microbes after ozone treatment

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for disinfecting or sanitizing, comprising:
delivering aqueous ozone having an ozone concentration of about 1 ppm to about 4 ppm to a sprayer at a pressure of about 200 psi to about 600 psi; and
spraying the pressurized aqueous ozone at a surface to reduce microbe populations on the surface.

2. The method of claim 1, further comprising collecting the aqueous ozone after spraying and irradiating the collected aqueous ozone with UV to destroy remaining ozone.

3. The method of claim 2, further comprising, heating the UV irradiated water.

4. The method of claim 3, further comprising rinsing a second surface with the heated and UV irradiated water.

5. The method of claim 1, wherein the surface is inside of a vessel.

6. The method of claim 1, wherein the ozone concentration is from 1 ppm to 4.5 ppm.

7. The method of claim 1, wherein the pressure is 200 psi to 600 psi.

8. The method of claim 1, wherein the pressure is 160 psi to 240 psi.

9. The method of claim 1, wherein the pressure is 540 psi to 660 psi.

10. The method of claim 1, wherein the ozone concentration is about 1 ppm to 4 ppm and the pressure is about 200 psi to about 600 psi.

11. The method of claim 10, wherein a temperature of the aqueous ozone is about 60° F. to about 85° F.

12. The method of claim 1, comprising spraying to reduce the populations of one of Escherichia coli, Pseudomonas aeruginosa, Staphylococcus aureus, or Salmonella enterica on the surface.

13. The method of claim 1, comprising reducing microbe populations by at least 2 log.

14. The method of claim 1, comprising reducing microbe populations by at least 3 log.

15. The method of claim 1, comprising reducing microbe populations by at least 4 log.

16. A system for disinfecting or sanitizing, comprising:
a storage tank containing aqueous ozone with an ozone concentration from about 1 ppm to about 4 ppm;
an ozone generator connected to the storage tank; and
a pump connected to the storage tank, wherein the pump produces a pressure of about 200 psi to about 600 psi.

17. The system of claim 16, further comprising a sprayer connected to the pump.

18. The system of claim 16, wherein the sprayer is a permanent fixture of a vessel.

19. The system of claim 16, wherein the sprayer is a removable fixture of a vessel.

20. The system of claim 16, wherein the storage tank has a recirculation loop.

21. The system of claim 16, comprising an ozone concentration meter that measures the ozone concentration of the aqueous ozone in the storage tank.

22. The system of claim 16, comprising a heater or cooler set to maintain a temperature of the aqueous ozone from about 60° F. to about 85° F.
